(19) United States
(12) Patent Application Publication
(10) Pub. No.: US 2021/0244930 A1
(43) Pub. Date: Aug. 12, 2021

(54) VALVED HANDLE ASSEMBLY HAVING A MOVABLE RING

(71) Applicant: KONINKLIJKE PHILIPS N.V., EINDHOVEN (NL)

(72) Inventors: Tyson REMM, PEYTON, CO (US); Joseph Charles Pius GRIEBEL, JR., COLORADO SPRINGS, CO (US); James David CEZO, COLORADO SPRINGS, CO (US); Adam TSCHIDA, GREENFIELD, MA (US)

(21) Appl. No.: 17/271,223

(22) PCT Filed: Sep. 6, 2019
(86) PCT No.: PCT/EP2019/073804

§ 371 Date: Feb. 25, 2021

Related U.S. Application Data
(60) Provisional application No. 62/728,004, filed on Sep. 6, 2018.

(51) Int. Cl. A61M 39/02; A61M 39/06

(52) U.S. Cl. CPC A61M 39/0247; A61M 39/0613; A61M 2039/027; A61M 2039/0686; A61M 2039/0294; A61M 2039/062; A61M 2039/0673; A61M 2039/0273

(57) ABSTRACT

The present disclosure discussed a device, method and/or system for performing a sealing control for a vascular treatment system having a handle assembly. To perform the sealing control, a movable ring associated with the handle assembly is adjusted to at least one of: a first position to provide a minimum pressure seal between the catheter and the handle assembly such that the catheter is freely movable within the handle assembly, a second position to provide a predetermined amount of pressure seal between the catheter and the handle assembly such that the catheter is movable in frictional contact with the handle assembly, and a third position to provide a maximum pressure seal between the catheter and the handle assembly such that the catheter is unmovable within the handle assembly.

### CROSS-REFERENCE TO RELATED APPLICATION

None.

### FIELD OF THE DISCLOSURE

The present disclosure relates generally to devices, methods and systems associated with vascular devices and techniques to perform a sealing control for a vascular treatment system during a vascular surgical procedure.

### BACKGROUND

A vascular treatment system having a laser ablation catheter can be used to treat a blockage of a peripheral or coronary artery in a patient. For example, a buildup of atherosclerotic plaque or other deposits can be formed in the artery causing non-healing ulcers or other anomalies for the patient. During the vascular surgical procedure, the laser ablation catheter is manipulated to position a distal end of the laser ablation catheter at a target area in the artery. For easy manipulation, the laser ablation catheter is inserted into an outer sheath allowing a clinician to readily translate the laser ablation catheter within the sheath along a longitudinal axis of the sheath in forward and backward directions. Typically, a conventional valve (e.g., a hemostasis valve) comprises a housing which defines a passage through which a catheter can penetrate a compliant material to create a sealed condition and maintain hemostasis; this valve may also be used for continuous or intermittent irrigation of a liquid medium, such as a contrast solution comprising a biocompatible fluid (e.g., saline), into a gap between the laser ablation catheter and the sheath.

However, significant back bleeding or leakage can occur from an interface from the valve and/or between the outer sheath and the laser ablation catheter when an insufficient or inadequate seal is provided for the valve. For example, such leakage of pressurized saline from the valve can result in stagnation of blood within the catheter and can become a potential source of health risk, such as emboli, or impact the intended treatment provided by the catheter. Moreover, existing vascular treatment systems are unable to provide a user feedback about a quality of the seal and are also unable to perform an effective adjustment of the seal during the vascular surgical procedure.

### SUMMARY

Accordingly, there is a need for an enhanced device, method and/or system for efficiently performing a sealing control for a vascular treatment system. The present disclosure discusses such a device, method and/or system. An example of a use of the vascular treatment system in accordance with this disclosure includes a handle assembly having a movable ring configured to perform the sealing control between a catheter and the handle assembly.

The present disclosure presents a method of performing a sealing control for a vascular treatment system. The method includes providing a handle assembly configured to receive a catheter and connected to the vascular treatment system, and providing a movable ring associated with the handle assembly and configured for performing the sealing control between the catheter and the handle assembly, the sealing control performed by adjusting the movable ring to two or more positions to provide a distinct pressure seal based on a corresponding position of the movable ring.

The method according to the previous paragraph, wherein the adjusting the movable ring to the two or more positions comprises at least one of: adjusting the movable ring to a first position to provide a first pressure seal between the catheter and the handle assembly such that the catheter is freely movable within the handle assembly, adjusting the movable ring to a second position to provide a second pressure seal between the catheter and the handle assembly such that the catheter is movable in frictional contact with the handle assembly, and adjusting the movable ring to a third position to provide a third pressure seal between the catheter and the handle assembly such that the catheter is unmovable within the handle assembly.

The method according to any of the previous paragraphs, further comprising variably translating the movable ring relative to the handle assembly to facilitate axial movement of the movable ring with respect to the handle assembly.

The method according to any of the previous paragraphs, further comprising variably rotating the movable ring relative to the handle assembly to facilitate axial movement of the movable ring with respect to the handle assembly.

The method according to any of the previous paragraphs, wherein the movable ring is rotated by zero degree relative to the handle assembly when the movable ring is in the first position.

The method according to any of the previous paragraphs, wherein the movable ring is rotated by greater than zero degree but less than 180 degrees relative to the handle assembly when the movable ring is in the second position.

The method according to any of the previous paragraphs, wherein the movable ring is rotated by equal to or greater than 180 degrees relative to the handle assembly when the movable ring is in the third position.

The method according to any of the previous paragraphs, wherein the handle assembly includes a proximal section, and a distal section having the movable ring such that the proximal section and the distal section are able to be separated.

The method according to any of the previous paragraphs, wherein the first pressure seal provides a minimum pressure seal between the catheter and the handle assembly.

The method according to any of the previous paragraphs, wherein the second pressure seal provides a predetermined amount of pressure seal between the catheter and the handle assembly.

The method according to any of the previous paragraphs, wherein the third pressure seal provides a maximum pressure seal between the catheter and the handle assembly.

The present disclosure also presents a vascular treatment system including a handle assembly configured to receive a catheter, and a movable ring associated with the handle assembly and configured for performing a sealing control between the catheter and the handle assembly, wherein, to perform the sealing control, the movable ring is adjusted to two or more positions to provide a distinct pressure seal based on a corresponding position of the movable ring.

The system according to the previous paragraph, wherein the two or more positions include at least one of: a first position to provide a minimum pressure seal between the catheter and the handle assembly such that the catheter is freely movable within the handle assembly, a second position to provide a predetermined amount of pressure seal between the catheter and the handle assembly such that the catheter is movable in frictional contact with the handle assembly, and a third position to provide a maximum pressure seal between the catheter and the handle assembly such that the catheter is unmovable within the handle assembly.

The system according to any of the previous paragraphs, wherein the movable ring is variably translatable relative to the handle assembly to facilitate axial movement of the movable ring with respect to the handle assembly.

The system according to any of the previous paragraphs, wherein the movable ring is variably rotatable relative to the handle assembly to facilitate axial movement of the movable ring with respect to the handle assembly.

The system according to any of the previous paragraphs, wherein the first position, the second position, and the third position of the movable ring is determined based on a rotational angle of the movable ring relative to the handle assembly.

The system according to any of the previous paragraphs, wherein the handle assembly includes a variable pitch thread configured to accommodate the axial movement of the movable ring.

The system according to any of the previous paragraphs, wherein the first position, the second position, and the third position of the movable ring is determined based on a different position in the variable pitch thread.

The system according to any of the previous paragraphs, wherein the handle assembly includes a variable cam configured to accommodate the axial movement of the movable ring.

The system according to any of the previous paragraphs, wherein the first position, the second position, and the third position of the movable ring is determined based on a different position of the variable cam.

The system according to any of the previous paragraphs, wherein the handle assembly includes a proximal section, and a distal section having the movable ring such that the proximal section and the distal section are able to be separated.

The system according to any of the previous paragraphs, wherein the movable ring of the distal section is axially movable relative to the handle assembly for performing the sealing control.

The system according to any of the previous paragraphs, wherein the movable ring of the distal section includes a pin extending from an inner wall of the movable ring.

The system according to any of the previous paragraphs, wherein the pin of the movable ring is at least partially inserted into a thread of a valve disposed in the distal section of the handle assembly for facilitating axial movement of the movable ring relative to the handle assembly.

The system according to any of the previous paragraphs, wherein the thread contains physical indicators when in a position.

The system according to any of the previous paragraphs, wherein the handle assembly transitioning between an engaged mode to a disengaged mode enables the moveable ring to access one or more previously blocked positions.

The system according to any of the previous paragraphs, wherein the handle assembly transition between an engaged mode to a disengaged mode disables one or more positions of the moveable ring.

The present disclosure also presents a method of performing a sealing control for a vascular treatment system. The method includes providing a movable ring configured for performing the sealing control for the catheter, the sealing control performed by adjusting the movable ring to two or more positions to provide a distinct pressure seal based on a corresponding position of the movable ring.

The method according to the previous paragraph, wherein the adjusting the movable ring to the two or more positions comprises at least one of: adjusting the movable ring to a first position to provide a first pressure seal for the catheter such that the catheter is freely movable, adjusting the movable ring to a second position to provide a second pressure seal for the catheter such that the catheter is movable in frictional contact; and adjusting the movable ring to a third position to provide a third pressure seal for the catheter such that the catheter is unmovable.

The method according to any of the previous paragraphs, further comprising variably translating the movable ring to facilitate axial movement of the movable ring.

The method according to any of the previous paragraphs, further comprising variably rotating the movable ring to facilitate axial movement of the movable ring.

The method according to any of the previous paragraphs, wherein the movable ring is rotated by zero degree when the movable ring is in the first position.

The method according to any of the previous paragraphs, wherein the movable ring is rotated by greater than zero degree but less than 180 degrees when the movable ring is in the second position.

The method according to any of the previous paragraphs, wherein the movable ring is rotated by 180 degrees when the movable ring is in the third position.

The method according to any of the previous paragraphs, wherein the first pressure seal provides a minimum pressure seal for the catheter.

The method according to any of the previous paragraphs, wherein the second pressure seal provides a predetermined amount of pressure seal for the catheter.

The method according to any of the previous paragraphs, wherein the third pressure seal provides a maximum pressure seal for the catheter.

The present disclosure also presents a vascular treatment system including a movable ring configured for performing a sealing control for the catheter, wherein, to perform the sealing control, the movable ring is adjusted to two or more positions to provide a distinct pressure seal based on a corresponding position of the movable ring.

The system according to the previous paragraph, wherein the two or more positions includes at least one of: a first position to provide a minimum pressure seal for the catheter such that the catheter is freely movable; a second position to provide a predetermined amount of pressure seal for the catheter such that the catheter is movable in frictional contact; and a third position to provide a maximum pressure seal for the catheter such that the catheter is unmovable.

The system according to any of the previous paragraphs, wherein the movable ring is variably translatable to facilitate axial movement of the movable ring.

The system according to any of the previous paragraphs, wherein the movable ring is variably rotatable to facilitate axial movement of the movable ring.

The system according to any of the previous paragraphs, wherein the first position, the second position, and the third position of the movable ring is determined based on a rotational angle of the movable ring.

The system according to any of the previous paragraphs, wherein the vascular treatment system includes a valve having a variable pitch thread configured to accommodate the axial movement of the movable ring.

The system according to any of the previous paragraphs, wherein the first position, the second position, and the third position of the movable ring is determined based on a different position in the variable pitch thread.

The system according to any of the previous paragraphs, wherein the vascular treatment system includes a valve having a variable cam configured to accommodate the axial movement of the movable ring.

The system according to any of the previous paragraphs, wherein the first position, the second position, and the third position of the movable ring is determined based on a different position of the variable cam.

The system according to any of the previous paragraphs, wherein the movable ring is axially movable for performing the sealing control.

The system according to any of the previous paragraphs, wherein the movable ring includes a pin extending from an inner wall of the movable ring.

The system according to any of the previous paragraphs, wherein the pin of the movable ring is at least partially inserted into a thread of a valve for facilitating axial movement of the movable ring.

The system according to any of the previous paragraphs, wherein the thread contains at least one physical indicator when in the first position, the second position, or the third position.

The system of the present disclosure can be used with medical devices and/or other devices that include the insertion of a liquid between a catheter and an outer sheath, such as the devices disclosed in U.S. application Ser. No. 15/659,064, filed Jul. 25, 2017 entitled Laser-Induced Pressure Wave Emitting Catheter Sheath and the devices disclosed in U.S. Application Ser. No. 62/716,965, filed Aug. 9, 2018 entitled Treatment Mode Selection Systems and Laser Catheter Systems Including Same (Attorney Docket No. 487145.001123), all of which are hereby incorporated herein by reference in their entireties for all that they teach and for all purposes. These two patent applications disclose a laser catheter inserted within an outer sheath and inserting liquid therebetween, thereby requiring a seal, but other types of catheters may be inserted in an outer sheath and liquid introduced or injected therebetween. For example, other types of catheters may include liquid light guides or radio-frequency ablation catheters. Examples of a liquid light guide, or a catheter that contain a liquid light guide can be seen in U.S. application Ser. No. 11/923,488, filed Oct. 24, 2007 and U.S. application Ser. No. 12/254,254, filed Oct. 20, 2008, both of which are hereby incorporated herein by reference in their entireties for all that they teach and for all purposes.

Additionally, examples of other catheter systems can be seen in U.S. application Ser. No. 15/469,240, filed Mar. 24, 2017 (Attorney Docket No. 487145.000809), and U.S. Application Ser. No. 62/716,965, filed Aug. 9, 2018 entitled Treatment Mode Selection Systems and Laser Catheter Systems Including Same (Attorney Docket No. 487145.001123), all of which are hereby incorporated herein by reference in their entireties for all that they teach and for all purposes.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X₁-Xₙ, Y₁-Yₘ, and Z₁-Zₒ, the phrase is intended to refer to a single element selected from X, Y, and Z, a combination of elements selected from the same class (e.g., X₁ and X₂) as well as a combination of elements selected from two or more classes (e.g., Y₁ and Zₒ).

The term “a” or “an” entity refers to one or more of that entity. As such, the terms “a” (or “an”), “one or more” and “at least one” may be used interchangeably herein. It is also to be noted that the terms “comprising”, “including”, and “having” may be used interchangeably.

The term “means” as used herein shall be given its broadest possible interpretation in accordance with 35 U.S.C. Section 112(f). Accordingly, a claim incorporating the term “means” shall cover all structures, materials, or acts set forth herein, and all of the equivalents thereof. Further, the structures, materials or acts and the equivalents thereof shall include all those described in the summary, brief description of the drawings, detailed description, abstract, and claims themselves.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

### BRIEF DESCRIPTION OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure may be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 shows a plan view of a handle assembly of a vascular treatment system in an engaged mode according to the present disclosure.

FIG. 2 shows a plan view of the handle assembly of FIG. 1 in a disengaged mode according to the present disclosure.

FIG. 3 shows an enlarged plan view of a distal section and a proximal section of the handle assembly of FIG. 2 in the disengaged mode.

FIG. 4 shows an enlarged plan view of a movable ring and a valve associated with the handle assembly of FIG. 2.

FIG. 5 shows a graphical presentation of a relationship between rotational movement of the movable ring and a travel distance of the movable ring caused by the rotational movement.

It should be understood that the drawings are not necessarily to scale. In certain instances, details that are not necessary for an understanding of the disclosure or that render other details difficult to perceive may have been omitted. It should be understood, of course, that the disclosure is not necessarily limited to the particular embodiments illustrated herein.

### DETAILED DESCRIPTION

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

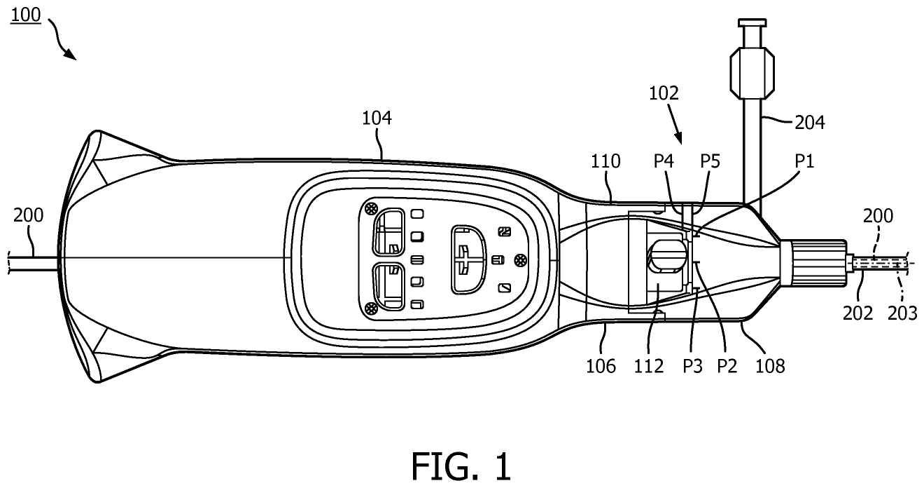

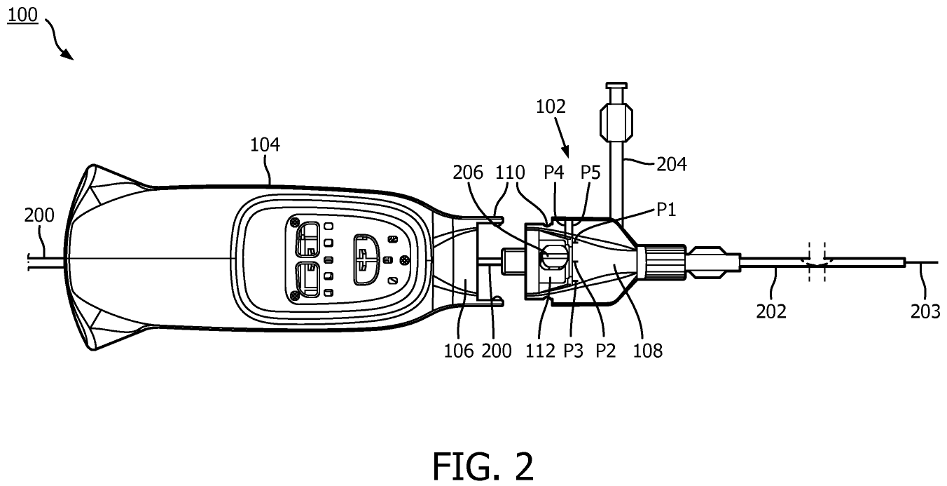

The present disclosure relates generally to devices, methods and systems associated with vascular devices and techniques to perform a sealing control for a vascular treatment system during a vascular surgical procedure. Referring to FIG. 1, there is shown an exemplary vascular treatment system 100 configured to perform the sealing control for the purpose of preventing or mitigating the leakage from the system 100 during the vascular surgical procedure. The system 100 illustrated in FIG. 1 includes a handle assembly 102. The handle assembly 102 can be operationally coupled to a motor drive unit (MDU) 104 for facilitating manipulation of a laser catheter 200 (FIG. 2) during the procedure. The handle assembly 102 receives the laser catheter 200 and transitions between an engaged mode (FIG. 1) and a disengaged mode (FIG. 2).

FIG. 1 shows the handle assembly 102 in the engaged mode. In this embodiment, the handle assembly 102 includes a proximal section 106 and a distal section 108. The engaged mode refers to a condition when the proximal section 106 and the distal section 108 are connected together using an attachment mechanism 110. For example, the attachment mechanism 110 can be an interlocking device having a snap connector, a threaded twist connector, a push button connector, or the like. Other configurations, such as magnetic or spring activated connectors, are also contemplated. When the proximal section 106 and the distal section 108 are separated from each other, the handle assembly 102 is in the disengaged mode (FIG. 2). In use, the manipulation of the laser catheter 200 (FIG. 2) can be performed when the handle assembly is in the engaged and/or disengaged mode.

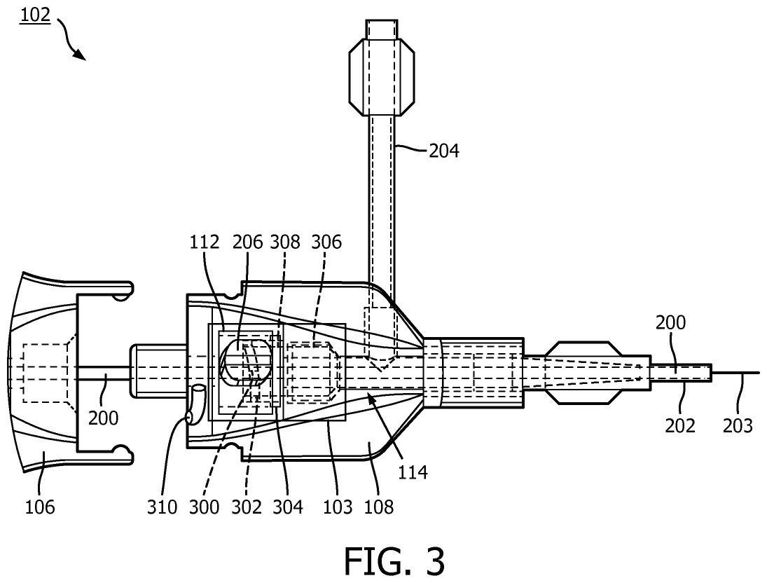

In FIG. 1, the proximal section 106 is fixedly attached to the MDU 104. In another embodiment, the proximal section 106 can be releasably attached to the MDU 104 using any suitable attachment mechanism discussed above. The distal section 108 can include a movable ring 112 and a valve assembly 103 (FIG. 3). The movable ring 112 can be an activation cap and is configured to perform the sealing control between the catheter 200 and the handle assembly 102. The valve assembly 103 is configured to create a sealed condition to maintain hemostasis and/or the irrigation of the liquid medium, such as the contrast solution comprising the biocompatible fluid (e.g., saline), for the system 100. The valve assembly 103 can be a hemostasis valve, a Tuohy-Borst adapter, a Y-shaped adapter, a bleed back control valve, and the like. In certain embodiments, the distal section 108 can also include a bleed back end 304 and/or a weep hole 310 to mitigate an unintentional leakage due to the back-flushing of the liquid medium injected into the valve body 114.

FIG. 2 shows the handle assembly 102 in the disengaged mode. In FIG. 2, the catheter 200 is slidably inserted into the valve assembly 103 (FIG. 3) and a tubular sheath 202 having a lumen therein and/or therethrough and configured to surround the catheter 200. For example, the catheter 200 can be inserted into the valve assembly 103 and within the lumen of the sheath 202, allowing a clinician to translate the catheter 200 within the valve assembly 103 and the sheath 202 along a longitudinal axis of the sheath 202 by manipulating the MDU 104 in forward and backward directions. The valve body 114 is fluidly coupled to the tubular sheath 202. Thus, during the vascular surgical procedure, the liquid medium can be supplied via an input port 204 of the valve body 114, and the supplied liquid medium can be injected into a gap between the catheter 200 and the sheath 202. In some embodiments, a guidewire 203 disposed within a lumen of the catheter 200 can be used to position a distal end of the catheter 200 in the target area within a vasculature of a subject.

To perform the sealing control, the movable ring 112 can be adjusted to at least one of three positions P1, P2, and P3. In this embodiment, a first position P1 provides no pressure or a minimum pressure seal between the catheter 200 and the handle assembly 102 such that the catheter 200 is freely movable, in both an axial (longitudinal) direction and a rotational direction, within the handle assembly 102 when the handle assembly is in the disengaged mode. A second position P2 provides a predetermined amount of pressure seal between the catheter 200 and the handle assembly 102 such that the catheter 200 is movable, in both an axial (longitudinal) direction and a rotational direction, in frictional contact with the handle assembly 102 when the handle assembly 102 is in the disengaged mode. A third position P3 provides a maximum pressure seal between the catheter 200 and the handle assembly 102 such that the catheter 200 is unmovable, in both an axial (longitudinal) direction and a rotational direction, within the handle assembly 102. In this embodiment, the first position P1, the second position P2, and the third position P3 of the movable ring 112 is determined based on a rotational angle of the movable ring 112 about a longitudinal axis of the movable ring 112 relative to the handle assembly 102.

In some situations, it is necessary to limit or restrict the number of positions available on the moveable ring 112 when in the engaged or disengaged mode. The engaged or disengaged mode of the handle assembly 102 can enable or disable the ability of the moveable ring 112 to access a position or combination of positions (P1-Pn where n denotes an integer). Although three positions P1, P2, P3 are shown herein for illustration purposes, any number of positions (e.g., four, five, six, and so forth, up to n positions) are also contemplated to suit different applications. This is accomplished through a physical interaction between the moveable ring 112 and one of either the proximal section 106 or distal section 108. This embodiment may include a protrusion from the proximal section which connects or physically limits the motion of the moveable ring 112 and therefore limits the number of positions available for the user to adjust the valve when in an engaged position. Disengaging the handle assembly 102, by disconnecting the distal section 108 from the proximal section 106 would then remove the physical limitation on the moveable ring 112. Thereby enabling the user to access all available positions on the moveable ring 112. In another embodiment of this would include a feature of the distal section 108 which interacts with the moveable ring 112 only when in the disengaged mode and limits the ability of the user to access certain positions on the moveable ring 112.

To facilitate rotational movement of the movable ring 112, the movable ring 112 includes a lever 206 extending from an outer surface or wall of the movable ring 112. For example, the lever 206 can be radially rotated clockwise or counterclockwise about the longitudinal axis of the movable ring 112. As a result, the movable ring 112 is selectively and variably rotatable relative to the handle assembly 102 to facilitate axial movement of the movable ring 112 with respect to the handle assembly 102. For example, when the lever 206 is moved from the first position P1 to the second position P2, the movable ring 112 moves forward toward a distal end of the distal section 108. Conversely, when the lever 206 is moved from the second position P2 to the first position P1, the movable ring 112 moves backward toward a proximal end of the distal section 108. Thus, the movable ring 112 of the distal section 108 is axially movable relative to the handle assembly 102 for performing the sealing control. Further, when the catheter 200 is movable within the handle assembly 102, the catheter can also move in an axial (longitudinal) direction and a rotational direction relative to the handle assembly 102.

Referring to FIG. 3, there is shown an enlarged plan view of the distal section 108 of the handle assembly 102. In this embodiment, the movable ring 112 includes a pin 300 extending from an inner surface or wall of the movable ring 112 and is configured to be at least partially inserted into a thread 302 of the valve body 114 disposed in the distal section 108 of the handle assembly 102 for facilitating axial movement of the movable ring 112 relative to the handle assembly 102. For example, the thread 302 can be a variable pitch thread configured to accommodate the axial movement of the movable ring 112. In this embodiment, the thread 302 is disposed on an outer surface or wall of a bleed back end 304 of the valve assembly 103 111. During the axial movement of the movable ring 112, the pin 300 travels along the thread 302 varying the position of the pin 300 in the thread 302. Thus, the first position P1, the second position P2, and the third position P3 of the movable ring 112 are determined based on different positions of the pin 300 in the thread 302.

Also included in the bleed back end 304 of the valve assembly 103 is a seal element 306 configured to provide radial sealing force around the catheter 200. In this embodiment, the radial sealing force can withstand an inner pressure in the valve assembly 103, e.g., up to approximately 300 pounds per square inch, caused by the injected liquid medium therein. The seal element 306 can be made of a resilient material, such as a rubber or plastic. Thus, the seal element 306 is deformable when compressed or depressed. In this example, the seal element 306 includes a flexible lumen therein and/or there through and is configured to surround the catheter 200.

In one embodiment, the movable ring 112 is variably rotated relative to the valve assembly 103 to facilitate axial movement of the movable ring with respect to the valve assembly 103 of the handle assembly 102. In one example, the movable ring 112 is configured to be rotatable clockwise or counterclockwise about the longitudinal axis of the movable ring 112 with respect to the valve assembly 103. In another embodiment, the movable ring 112 is variably translated in a longitudinal direction along the longitudinal axis of the movable ring 112 with respect to the valve assembly 103 of the handle assembly 102. In one example, the movable ring 112 is configured to be translatably or reciprocably moved forward or backward in the longitudinal direction along the longitudinal axis of the movable ring 112 with respect to the valve assembly 103. In certain situations, the translation movement of the movable ring 112 is achieved without rotating the movable ring 112 by slidingly moving the movable ring 112 in the longitudinal direction. In other situations, the translation movement of the movable ring 112 can also be achieved at least partially rotating the movable ring 112.

For example, the catheter 200 can be inserted into the valve assembly 103 and within the lumen of the seal element 306, allowing the translation movement of the catheter 200 within the valve assembly 114 and the sheath 202 during use. When the radial sealing force around the catheter 200 is insufficient, the leakage of the liquid medium from the valve body 114 can occur. To increase the radial sealing force around the catheter 200, the movable ring 112 can be rotated, for example, from the first position P1 to the second position P2. Conversely, to decrease the radial sealing force around the catheter 200, the movable ring 112 can be rotated, for example, from the second position P2 to the first position P1.

In another example, to increase the radial sealing force around the catheter 200, the movable ring 112 can be translated forward in the longitudinal direction along the longitudinal axis of the movable ring 112 with respect to the valve assembly 103, for example, from a forth position P4 to a fifth position P5 (FIGS. 1 and 2). Conversely, to decrease the radial sealing force around the catheter 200, the movable ring 112 can be translated backward in the longitudinal direction along the longitudinal axis of the movable ring 112 with respect to the valve assembly 103, for example, from the fifth position P5 to the fourth position P4 (FIGS. 1 and 2). Although two available translation positions P4 and P5 are shown for illustration purposes, any number of positions (e.g., three, four, five, etc.) can be utilized to suit different applications.

The compression of the seal element 306 can be achieved by a plunger portion 308 of the movable ring 112. In one embodiment, the plunger portion 308 has a cylindrical shape and is disposed near a central axis of the movable ring 112. When the movable ring 112 transitions from the first position P1 to the second position P2, the plunger portion 308 of the movable ring 112 advances toward the seal element 306 and firmly engages an outer surface of the seal element 306 to deform the flexible lumen surrounding the catheter 200. As a result, an inner diameter of the flexible lumen surrounding the catheter 200 decreases, thereby increasing the pressure force around the catheter 200. For example, if a 7 French sized laser catheter is to be used, the inner diameter of the flexible lumen can be approximately 0.071 inches under no compression. Other suitable inner diameters of the flexible lumen commensurate with the French sized laser catheters are also contemplated to suit different applications.

In use, as the movable ring 112 moves forward toward the distal end of the distal section 108, an inner surface or wall of the flexible lumen gradually engages an outer radial surface or wall of the catheter 200 under the action of deformation of the flexible lumen. As a result, a predetermined amount of applied pressure (e.g., a blood pressure or an injection pressure) is maintained in the valve assembly 103. Thus, it is advantageous that the leakage and/or the applied pressure can be readily controlled during the vascular surgical procedure.

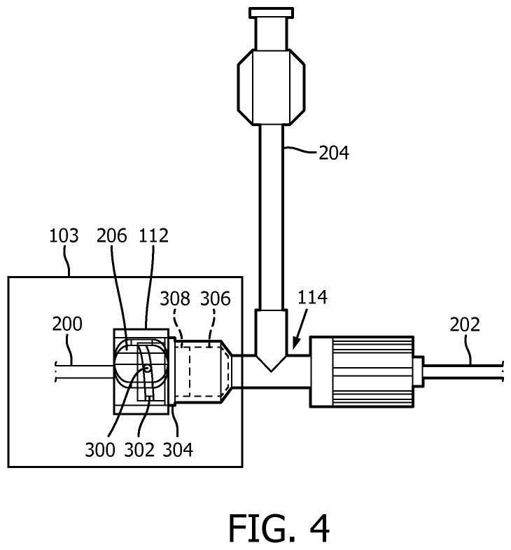

Referring to FIG. 4, there is shown an enlarged plan view of the movable ring 112 and the valve assembly 103 while the plunger portion 308 of the movable ring 112 is compressing the seal element 306 to increase the radial sealing force around the catheter 200. For example, when the movable ring 112 is rotated from the first position P1 to the second position P2, the plunger portion 308 directly engages the outer surface of the seal element 306 to decrease the inner diameter of the flexible lumen surrounding the catheter 200. As a result, the radial sealing force can be variably adjusted by the movable ring 112.

Referring now to FIGS. 2 and 4, the first position P1 refers to a condition where the movable ring 112 is rotated by zero degree relative to the handle assembly 102. When the movable ring 112 is adjusted to the first position P1, a first pressure seal is provided between the catheter 200 and the seal element 306 of the valve assembly 103 in the handle assembly 102. The first pressure seal causes the catheter 200 to be freely movable within the handle assembly 102. At the first position P1, the minimum pressure seal between the catheter 200 and the seal element 306 may cause the leakage of the liquid medium from the valve body 114. For example, the liquid medium can leak from the bleed back end 304 of the valve assembly 103.

The second position P2 refers to a condition where the movable ring 112 is rotated by greater than zero degree but less than 180 degrees relative to the handle assembly 102. When the movable ring 112 is adjusted to the second position P2, a second pressure seal is provided between the catheter 200 and the seal element 306 of the valve assembly 103 in the handle assembly 102. The second pressure seal causes the catheter 200 to be movable in frictional contact with the seal element 306 of the valve assembly 103 in the handle assembly 102. At the second position P2, the predetermined amount of pressure seal between the catheter 200 and the seal element 306 may cause the leakage of the liquid medium from the valve assembly 103. For example, the liquid medium can leak from the bleed back end 304 of the valve assembly 103.

The third position P3 refers to a condition where the movable ring 112 is rotated by 180 degrees relative to the handle assembly 102. When the movable ring 112 is adjusted to the third position P3, a third pressure seal is provided between the catheter 200 and the seal element 306 of the valve assembly 103 in the handle assembly 102. The third pressure seal creates a complete seal and causes the catheter 200 to be unmovable within the handle assembly 102. At the third position P3, the maximum pressure seal between the catheter 200 and the seal element 306 may cause no leakage of the liquid medium from the valve assembly 103. For example, the liquid medium does not leak from the bleed back end 304 of the valve assembly 103.

In another embodiment, the third pressure seal can be provided without the catheter 200. For example, when the catheter 200 is completely retracted from the handle assembly 102, the third pressure seal provides the complete seal in the flexible lumen of the seal element 306 so that the pressure in the valve assembly 103 is maintained without the catheter 200. Although three available rotation positions P1, P2, and P3 are illustrated herein for the movable ring 112, other suitable discrete or continuous positions (e.g., four, five, six, or more discrete positions) are also contemplated to suit different applications.

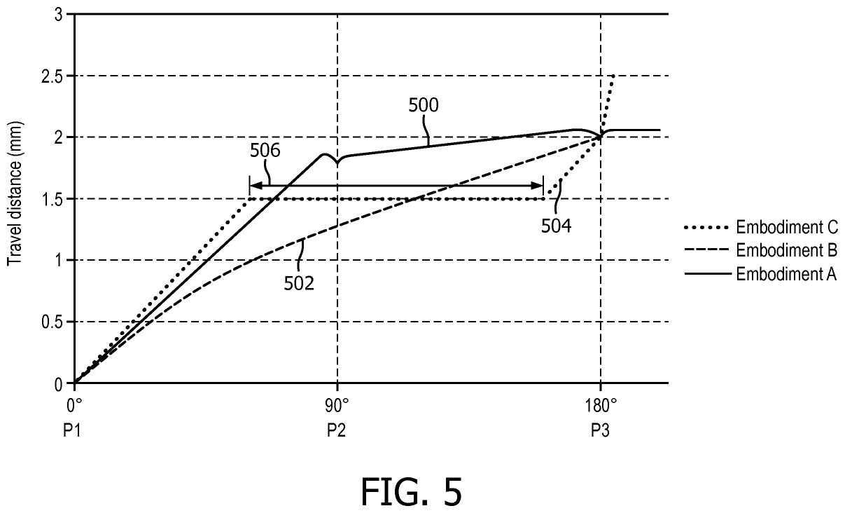

Referring now to FIG. 5, there is shown a graphical presentation of a relationship between rotational movement of the movable ring 112 and a travel distance of the movable ring 112 caused by the rotational movement. In FIG. 5, an X-axis represents a current position of the movable ring 112 (e.g., P1, P2, P3) and a corresponding degree of rotation (e.g., 0 degree, 90 degrees, 180 degrees), and a Y-axis represents a travel distance (e.g., 0-3 millimeters) of the movable ring 112 along the longitudinal axis of the movable ring 112 in relation to the current position of the movable ring 112. A path of the thread 302 (FIG. 4) of the valve assembly 103 can vary to accomplish a different radial sealing force of the valve assembly 103. In this embodiment, three positions P1, P2, and P3 are shown for the valve assembly 103 to perform three distinct functions. In FIG. 5, the graph shows these three (3) positions, namely P1, P2, and P3 as being equally spaced along a 180-degree path of rotation. In other embodiments, a number of positions can be one or more (e.g., one, two, three, four, etc.) and can be spaced over a range of 10-720 degrees. In yet other embodiments, the range can be between zero and 360 degrees.

A spacing between adjacent positions can be evenly distributed or unevenly spaced depending on the application. In FIG. 5, a total compression of the seal element 306 of the valve assembly 103 is depicted as approximately 2 millimeters in the graph. However, in various embodiments, the total compression can vary depending on a length and/or a thickness of the valve assembly 103. Three unique thread paths (Embodiment A, Embodiment B, and Embodiment C) are shown in FIG. 5. Any combinations of the features related to Embodiment A, Embodiment B, and Embodiment C are also contemplated to suit different applications.

Embodiment A shows the use of a variable cam for activation of the movable ring 112. For example, the handle assembly 102 can include the variable cam configured to accommodate the axial movement of the movable ring 112. The first position P1, the second position P2, and the third position P3 of the movable ring 112 can be determined based on a different position of the variable cam. Other suitable configurations, such as bumps or protrusions, can also be used to determine the position of the movable ring 112 in lieu of the variable cam.

A curved-line segment 500 associated with Embodiment A shows a profile that begins with a steeper pitch to compress the sealing element 306 over a 90-degree rotation to reach the second position P2. The handle 206 can then be rotated to the third position P3 to achieve full compression of the sealing element 306. A rotation greater than 180 degrees does not increase compression of the valve assembly 103. In this embodiment, the thread 302 can have one or more detents (e.g., compression in the path of the thread 302) to give a user feedback (e.g., a tactile or audible indication) when reaching the discrete positions along the path and to aid the valve assembly 103 in maintaining its current position.

The detents, bumps, or other suitable configurations, which can be used for activation of the movable ring 112 to determine discrete position (P1, P2, or P3), can interact with either the thread 302 and pin 300, or distal section 108 and moveable ring 112, or a combination thereof. Where the bumps or detents can be a feature of the movable ring 112 and engage the distal section 108 at discrete points along the rotational path.

Embodiment B shows the use of a smooth transitional path of the thread 302 between pitches along the rotation of the movable ring 112. In this embodiment, the current position is maintained by an interaction of the movable ring 112 and the distal section 108 as shown in FIGS. 2-4. A curved-line segment 502 associated with Embodiment B shows the profile representing a consistent increase of the travel distance of the movable ring 112 as the current position of the movable ring 112 changes. A variation in the path of the thread 302 can be configured to produce a constant torque to rotate the movable ring 112 during the 180 degrees of rotation. As the sealing element 306 is compressed, the sealing force to compress the sealing element 306 also changes. A pitch of the path of the thread 302 can be configured such that no or minimal change of the sealing force or the rotational torque is detected during operation. In some embodiments, the pitch can be constant and continuous. In certain embodiments, the discrete positions of the valve assembly 103 can also be utilized.

Embodiment C shows the use of a variable pitch in the path of the thread 302. A curved-line segment 504 associated with Embodiment C shows a section 506 of the profile where a portion of the rotation of the movable ring 112 causes no additional compression in the valve assembly 103 during the rotation. This enables a window of rotation where the sealing force of the valve assembly 103 is unchanged or maintained, functionally providing a window of rotation at P2. In this embodiment, the pitch can be constant and non-continuous. In certain embodiments, the pitches can be different or variable. In this embodiment, the pitch increases after 180 degrees of rotation to allow the movable ring 112 to be rotated past the third position P3, but to enable a return of the movable ring 112 to the third position P3. For example, when the movable ring 112 is released, the movable ring 112 can resiliently return to the third position P3 under the action of the sealing element 306 overcoming the compression force caused by the movable ring 112.

The foregoing discussion has been presented for purposes of illustration and description. The foregoing is not intended to limit the disclosure to the form or forms disclosed herein. In the foregoing Summary for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the description has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

1. A method of performing a sealing control for a vascular treatment system, the method comprising:
providing a handle assembly configured to receive a catheter and connected to the vascular treatment system; and
providing a movable ring associated with the handle assembly and configured for performing the sealing control between the catheter and the handle assembly, the sealing control performed by adjusting the movable ring to two or more positions to provide a distinct pressure seal based on a corresponding position of the movable ring;
wherein the adjusting the movable ring to the two or more positions comprises at least one of:
adjusting the movable ring to a first position to provide a first pressure seal between the catheter and the handle assembly such that the catheter is freely movable within the handle assembly;
adjusting the movable ring to a second position to provide a second pressure seal between the catheter and the handle assembly such that the catheter is movable in frictional contact with the handle assembly; and
adjusting the movable ring to a third position to provide a third pressure seal between the catheter and the handle assembly such that the catheter is unmovable within the handle assembly;
wherein the method further comprises variably rotating the movable ring relative to the handle assembly to facilitate axial movement of the movable ring with respect to the handle assembly;
wherein a plunger portion of the movable ring compresses a seal element to increase a radial sealing force around the catheter.

2.-4. (canceled)

5. The method of claim 1, wherein the movable ring is rotated by zero degree relative to the handle assembly when the movable ring is in the first position.

6. The method of claim 1, wherein the movable ring is rotated by greater than zero degree but less than 180 degrees relative to the handle assembly when the movable ring is in the second position.

7. The method of claim 1, wherein the movable ring is rotated by equal to or greater than 180 degrees relative to the handle assembly when the movable ring is in the third position.

8. (canceled)

9. The method of claim 1, wherein the first pressure seal provides a minimum pressure seal between the catheter and the handle assembly.

10. The method of claim 1, wherein the second pressure seal provides a predetermined amount of pressure seal between the catheter and the handle assembly.

11. The method of claim 1, wherein the third pressure seal provides a maximum pressure seal between the catheter and the handle assembly.

12. A vascular treatment system comprising:
a handle assembly configured to receive a catheter; and
a movable ring associated with the handle assembly and configured for performing a sealing control between the catheter and the handle assembly, wherein, to perform the sealing control, the movable ring is adjusted to two or more positions to provide a distinct pressure seal based on a corresponding position of the movable ring;
wherein the two or more positions include at least one of:
a first position to provide a minimum pressure seal between the catheter and the handle assembly such that the catheter is freely movable within the handle assembly;
a second position to provide a predetermined amount of pressure seal between the catheter and the handle assembly such that the catheter is movable in frictional contact with the handle assembly; and
a third position to provide a maximum pressure seal between the catheter and the handle assembly such that the catheter is unmovable within the handle assembly;
characterized in that the movable ring is variably rotatable relative to the handle assembly to facilitate axial movement of the movable ring with respect to the handle assembly;
wherein a plunger portion of the movable ring is configured to compress a seal element to increase a radial sealing force around the catheter.

13.-15. (canceled)

16. The system of claim 12, wherein the first position, the second position, and the third position of the movable ring is determined based on a rotational angle of the movable ring relative to the handle assembly.

17. The system of claim 12, wherein the handle assembly includes a variable pitch thread configured to accommodate the axial movement of the movable ring.

18. The system of claim 17, wherein the first position, the second position, and the third position of the movable ring is determined based on a different position in the variable pitch thread.

19. The system of claim 12, wherein the handle assembly includes a variable cam configured to accommodate the axial movement of the movable ring.

20. The system of claim 19, wherein the first position, the second position, and the third position of the movable ring is determined based on a different position of the variable cam.

21. The system of claim 12, wherein the handle assembly includes a proximal section, and a distal section having the movable ring such that the proximal section and the distal section are able to be separated;
wherein the movable ring of the distal section is axially movable relative to the handle assembly for performing the sealing control;
wherein the movable ring of the distal section includes a pin extending from an inner wall of the movable ring;
wherein the pin of the movable ring is at least partially inserted into a thread of a valve disposed in the distal section of the handle assembly for facilitating axial movement of the movable ring relative to the handle assembly; and
wherein the thread contains physical indicators when in a position.

22.-25. (canceled)

26. The system of claim 12, wherein the handle assembly transitioning between an engaged mode to a disengaged mode enables the moveable ring to access one or more previously blocked positions or disables one or more positions of the moveable ring.

27.-50. (canceled)
